(19) United States
(12) Patent Application Publication
(10) Pub. No.: US 2023/0320692 A1
(43) Pub. Date: Oct. 12, 2023

(54) METHOD FOR TRANSESOPHOGEAL ECHOCARDIOGRAPHY FOR CARDIOPULMONARY RESUSCITATION AND TREATMENT OF SHOCK

(71) Applicant: Ryan Giorgetti, Brooklyn, NY (US)

(72) Inventor: Ryan Giorgetti, Brooklyn, NY (US)

(21) Appl. No.: 17/658,751

(22) Filed: Apr. 11, 2022

(51) Int. Cl. A61B 8/08; A61B 8/00; A61B 8/12

(52) U.S. Cl. CPC A61B 8/0883; A61B 8/12; A61B 8/483; A61B 8/4245

(57) ABSTRACT

A method is provided of treating cardiac arrest or shock in a patient without effective cardiac output and being administered chest compressions to maintain blood flow during cardiac arrest. The method employs transesophogeal echocardiography (TEE) used in a twelve-view examination of standard TEE views (RESCUE TEE) performed in sequence to minimize problem movement and provide a logical flow of views and information. The method allows for rapid monitoring of intra arrest pathology and adjusting chest compressions as necessary to optimize blood flow and initiating other treatments to stabilize the patient.

### FIELD OF THE INVENTION

This invention pertains to methods of using transesophogeal echocardiography (TEE) in the treatment of cardiac arrest and shock using a specific sequence of TEE views.

### BACKGROUND

Point of care ultrasound (POCUS) is a valuable tool for the management of shock. POCUS can be used to make immediate patient-care decisions to evaluate an emergency medical condition, in settings such as an emergency department, critical care unit, ambulance, or combat zone. Emergency POCUS can quickly diagnose certain injuries or pathologic conditions where other diagnostic methods either take too long or would introduce greater risk to the patient, for example by requiring transporting the person away from closely monitored setting or exposing them to ionizing radiation and/or intravenous contrast agents. In some cases, POCUS can be used to visualize pathologic conditions that cannot be easily visualized using other techniques.

With respect to visualizations of the heart with ultrasonography, two techniques are in common use, transthoracic echocardiography (TTE) and transesophogeal echocardiography (TEE). TTE is conventional but suffers from several limitations. Poor acoustic windows may lead to misinterpretation of imaging and prolonged interruption of cardiopulmonary resuscitation (CPR) during pulse checks may negatively affect cardiac arrest resuscitation.[1] Transesophageal echocardiography (TEE) overcomes these limitations. In cardiac arrest, ultrasonography has been shown to accurately identify the presence or lack of intrinsic cardiac activity and, in some cases, the cause of cardiac arrest, including pulmonary embolism, pericardial tamponade, aortic dissection, myocardial infarction, valve failure/dysfunction, left ventricular failure, right ventricular failure, left atrial/ventricular outflow obstruction, hypertrophic obstructive cardiomyopathy, or hypovolemia.[1]

TEE has conventionally been used for an assessment of the cardiac source of embolism; suspected endocarditis, especially in a patient with prosthetic valve; suspected prosthetic valve dysfunction; an assessment of the thoracic aorta and other vessels (pulmonary trunk and arteries, pulmonary veins, coronary arteries); an assessment of mitral regurgitation; an assessment prior to valvular repairs and closures of septal defects; congenital heart defects; intraoperative monitoring of cardiac or percutaneous interventions, including ablation; non-diagnostic transthoracic examination, especially in patients after cardiac surgeries[2]. None of these indications pertain to emergency procedures. A review has been published of POCUS in cardiac arrest, which always an emergency, but the reviewers noted that as of August 2019, only TTE has been reported.[3]

Several general discussions of TEE have been reported.[4]-[6] In particular, Shanewise (ref. [4]) lists a series of standardized views in TEE. None discuss the use of TEE in emergency settings or provide the specific sequence of views of this invention.

### SUMMARY OF THE INVENTION

This method provides a means of rapidly assessing the cardiac status of a person in cardiac arrest, i.e., with a heart that has stopped. There are many causes of cardiac arrest, and a rapid assessment of the cause can lead to more effective treatments and increased likelihood of survival. Any situation involving cardiac arrest is an emergency; assessments and treatments must be performed rapidly.

Accordingly, a method is provided of visualizing cardiac arrest or shock in a patient without effective cardiac output and being administered chest compressions to maintain blood flow during cardiac arrest in order to rapidly identify the etiology of the cardiac arrest or shock and administer treatments to the patient. The method employs transesophogeal echocardiography (TEE) used in a twelve-view examination of standard TEE views performed in a specific sequence to minimize probe movement and provide a logical flow of views and information. We term this the “RESCUE (Rapid Evaluation in Shock and Cardiac arrest Using Emergent) TEE Protocol.” The method allows for rapid evaluation for intra arrest pathology and adjusting chest compressions as necessary to optimize blood flow and initiating other treatments to stabilize the patient.

### DESCRIPTION OF THE DRAWINGS

FIG. 1 is a drawing of a typical TEE apparatus including the probe.

FIGS. 2 a-d is an illustration of the types of motion used in TEE.

FIGS. 3 a-c illustrate conventions of the sonographic image displays used in TEE. Abbreviations: LA, Left atrium; LV, left ventricle; RV, right ventricle. FIG. 3d shows cross-sectional views of the heart from various positions of the transducer. FIG. 3d includes markings showing the corresponding views in FIG. 4 for the four cross-sectional views in FIG. 3d.

FIGS. 4 a-o shows fifteen cross-sectional views of the inventive transesophageal echocardiographic examination. Approximate multiplane angle is indicated by the icon adjacent to each view. Abbreviations: ME, Mid esophageal; LAX, long axis; TG, transgastric; SAX, short axis; AV, aortic valve; RV, right ventricle; asc , ascending; desc, descending; UE, upper esophageal.

### DETAILED DESCRIPTION

A method is provided herein for use of transesophogeal echocardiography (TEE) in emergency departments and critical care facilities for resuscitation of patients in acute cardiac distress from cardiac arrest due to conditions including pulmonary embolism, pericardial tamponade, aortic dissection, myocardial infarction, valvular failure/dysfunction, left ventricular failure, right ventricular failure, left atrial/ventricular outflow obstruction, or hypertrophic obstructive cardiomyopathy, or hypovolemia. Accurate identification of the specific cause of cardiac arrest should lead to more specific treatments and improved patient outcomes.

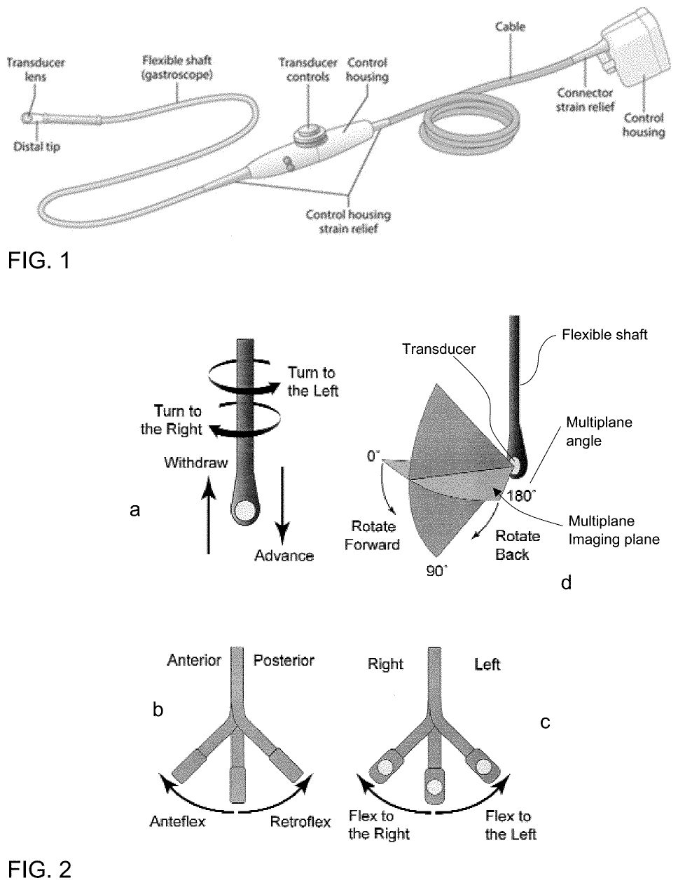

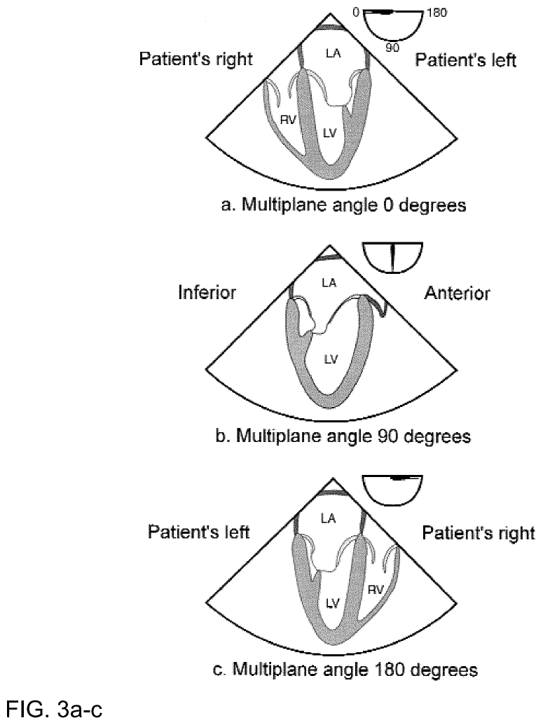

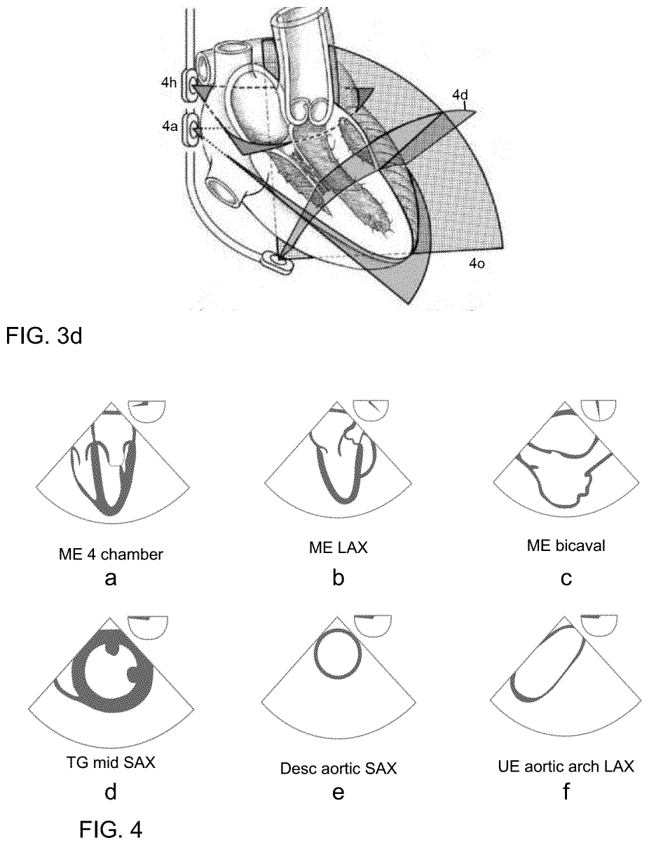

TEE procedures are performed with a special probe and transducer that can be inserted into the esophagus of a patient in distress and moved in several orientations in situ. FIG. 1 shows an exemplary probe. FIG. 2 shows manipulations that the probe can perform. FIG. 2a indicates that the probe can be advanced or withdrawn in the esophagus and stomach. FIG. 2b shows anteflexion and retroflexion, FIG. 2c show flexing to the right and left. FIG. 2d shows the multiplane imaging plane. The multiplane angle is adjustable by the rotational movement shown in FIG. 2a. FIG. 3 shows conventions of image display in TEE in the heart. Transducer location and the near field (vertex) of the image sector are at the top of the display screen and far field is at the bottom. FIG. 3a shows Image orientation at a multiplane angle of 0 degrees. FIG. 3b shows image orientation at a multiplane angle of 90 degrees. FIG. 3c shows image orientation at multiplane angle of 180 degrees

The inventive method provides a method of visualizing cardiac arrest or shock in a patient without effective cardiac output and being administered chest compressions to maintain blood flow during cardiac arrest in order to rapidly identify the etiology of the cardiac arrest or shock and administer appropriate treatments to the patient. The method includes an initial assessment of pathology using a basic four TEE view exam followed by an aortic sweep maneuver with eight additional TEE views. The inventive method employs a specific sequence of views designed to minimize probe movement, provide a systematic flow of views, and maximize information to the clinician. We term this the “RESCUE (Rapid Evaluation in Shock and Cardiac arrest Using Emergent) TEE Protocol.” The clinician can then respond with appropriate treatments as described below.

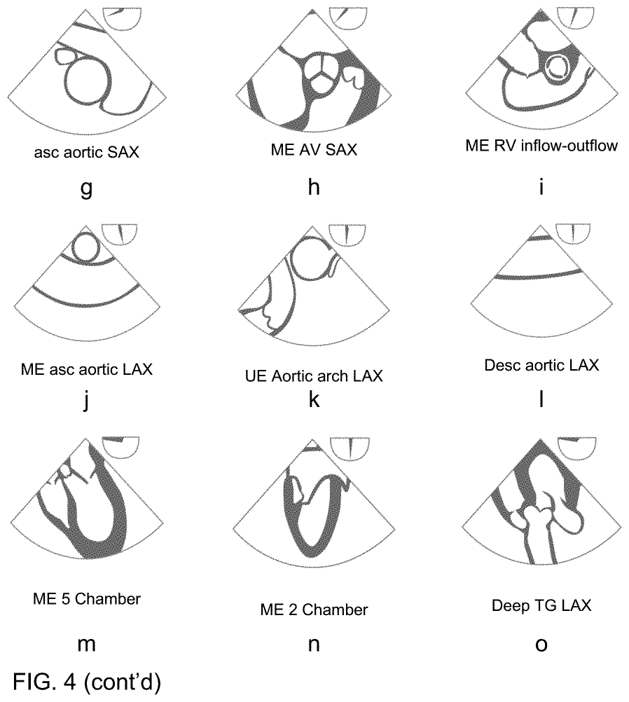

The views are shown in FIG. 4. The basic four view exam is FIGS. 4a-d, showing the mid-esophageal 4 chamber view (FIG. 4a), the mid-esophageal long axis view (FIG. 4b), the mid-esophageal bi-caval view (FIG. 4c), and the trans-gastric mid-short axis view (FIG. 4d).

The aortic sweep maneuver comprises eight additional views shown in FIG. 4e-l. The descending thoracic aorta is followed back to the aortic valve in zero degrees for the “0-degree sweep”. The multiplane angle is then increased to ninety degrees to obtain the ascending aorta and is followed back to the descending thoracic aorta for the “90-degree sweep”. While moving from 0-degrees to 90-degress at the level of the aortic valve, the examiner will pass through two standard views. These inter-sweep views are the mid-esophageal aortic valve short axis (30 degrees) and the mid esophageal right ventricular inflow-outflow (60-70 degrees). Therefore, in the process of following the aorta during the sweeps, the examiner will pass through eight additional standard TEE views. These views are the descending thoracic aorta short axis view (FIG. 4e), the upper esophageal aortic arch long axis view (FIG. 4f), the mid-esophageal ascending aorta short axis view (FIG. 4g), the mid-esophageal aortic valve short axis view (FIG. 4h), the mid-esophageal right ventricular inflow-outflow view (FIG. 4i), the mid-esophageal ascending aorta long axis view (FIG. 4j), the upper esophageal aortic arch short axis view (FIG. 4k), and the descending aorta long axis view (FIG. 4l). With this sequence of views, critical structures such as the aorta, pulmonary artery, valves, left ventricle, and right ventricle become visible in multiple planes. This allows for a thorough evaluation of intra-arrest pathology.

After the assessment for pathology is completed, the focus of the exam shifts to continuous hemodynamic monitoring. The sonographic compression vector is monitored to ensure effective compression of the left ventricle. Should the area of maximum compression be found over the ascending aorta, aortic valve or left ventricular outflow tract (LVOT), chest compressions can be repositioned to a more effective location. The pulse check periods are also monitored for myocardial activity. Visual confirmation of organized activity, ventricular fibrillation or cardiac standstill can be made in seconds and shorten pauses in CPR. In the case of shock, three additional views are used that allow for hemodynamic measurements using doppler ultrasound. These views are the mid-esophageal 5 chamber view (FIG. 4m), the mid-esophageal 2 chamber view (FIG. 4n), and the deep trans-gastric 5 chamber view (FIG. 4o). These additional views may be skipped during the intra-arrest period.

The inventive method allows for a systematic and rapid evaluation in cardiac arrest and shock. This can help with a diagnosis of why the heart stopped, and identify etiologies such as blood clots, valvular issues, volumetric issues, or suggests an electrical/metabolic issue if structural causes with the heart have been ruled out. The RESCUE TEE information obtained by the inventive method can direct treatments, for example clot busting drugs if a clot is identified. Selection of appropriate medications can be guided by the inventive method. Other treatments that may be effectively guided by the inventive method include extracorporeal cardiopulmonary resuscitation (ECPR), extracorporeal membrane oxygenation (ECMO), positional guidance of percutaneous left ventricular assist devices (IMPELLA catheter), right heart catheter placement, transvenous pacemaker placement and decreasing the risk of cardioversion for abnormal heart rhythms such as atrial fibrillation.

Although transthoracic echocardiography (TTE) may be highly useful to a clinician, there are several physical and logistical limitations to TTE that affect its usefulness in critical care settings. Limited sonographic acoustic windows may impair the ability to adequately assess cardiac function. In addition, prolonged breaks during CPR to allow for assessment of cardiac function hinders the resuscitative effort. The consequences of prolonged breaks in CPR cannot be overstated. Brief interruptions have been shown to reduce perfusion pressure rapidly. This often requires up to one minute of compressions to restore the perfusion pressure to its level prior to the break.

TEE offers the benefit of imaging superior to that obtained from TTE without limitations of impaired acoustic windows or pauses in CPR. From its position in the esophagus directly adjacent to the heart, the close proximity of the TEE probe allows the examiner to consistently obtain high quality image resolution. In addition, the TEE probe can be left in place during CPR to provide real-time, continuous monitoring of cardiac function without interrupting compressions. This allows for ongoing assessment of CPR quality. Improper positioning of CPR compressions may be identified by TEE images demonstrating the sonographic compression vector centered over the LVOT, aortic valve, or ascending aorta. Compressions can be immediately repositioned leading to improvement in perfusion. In addition, TEE has been shown to identify fine ventricular fibrillation during pulse checks when the monitor shows asystole. This leads to a different treatment algorithm and may have a significant impact on the course of the resuscitation.

The ability of RESCUE TEE to provide feedback on CPR performance in real time and potentially identify the etiology of the cardiac arrest make it an ideal tool for the clinician in emergency and critical care settings.

In the event of a conflict between the teachings of this patent application and those of any incorporated or cited document, the teachings of this patent application control.

### REFERENCES

(1) Giorgetti, R.; Chiricolo, G.; Melniker, L.; Calaf, C.; Gaeta, T. RESCUE Transesophageal Echocardiography for Monitoring of Mechanical Chest Compressions and Guidance for Extracorporeal Cardiopulmonary Resuscitation Cannulation in Refractory Cardiac Arrest. Journal of Clinical Ultrasound 2020, 48 (3), 184-187. https://doi.org/https://doi.org/10.1002/jcu.22788.
(2) Szyszka, A.; Plońska-Gościniak, E. Transesophageal Echocardiography. Journal of Ultrasonography 2019, 19 (76), 62-65. https://doi.org/10.15557/JoU.2019.0009.
(3) Kedan, I.; Ciozda, W.; Palatinus, J. A.; Palatinus, H. N.; Kimchi, A. Prognostic Value of Point-of-Care Ultrasound during Cardiac Arrest: A Systematic Review. Cardiovascular Ultrasound 2020, 18 (1), 1. https://doi.org/10.1186/s12947-020-0185-8.
(4) Shanewise, J. S.; Cheung, A. T.; Aronson, S.; Stewart, W. J.; Weiss, R. L.; Mark, J. B.; Savage, R. M.; Sears-Rogan, P.; Mathew, J. P.; Quinones, M. A.; Cahalan, M. K.; Savino, J. S. ASE/SCA Guidelines for Performing a Comprehensive Intraoperative Multiplane Transesophageal Echocardiography Examination: Recommendations of the American Society of Echocardiography Council for Intraoperative Echocardiography and the Society of Cardiovascular Anesthesiologists Task Force for Certification in Perioperative Transesophageal Echocardiography. Journal of the American Society of Echocardiography 1999, 12 (10), 884-900. https://doi.org/10.1016/S0894-7317(99)70199-9.
(5) Benenstein, R. J. BASIC TEE PROTOCOL; 2011.
(6) Cloutier, J.; Lewis, D. Introduction to Transesophageal Echo-Basic Technique https://syhem.ca/introduction-to-transesophageal-echo-basic-technique/(accessed 2022 -03 -08).

1. A method of visualizing cardiac arrest or shock in a patient without effective cardiac output and being administered chest compressions to maintain blood flow during cardiac arrest in order to rapidly identify the etiology of the cardiac arrest or shock and administer treatments to the patient, comprising inserting a transesophogeal echocardiography transducer into the esophagus of a patient experiencing cardiac arrest and performing a basic four view examination followed by an aortic sweep maneuver wherein the following views are obtained in sequence: mid-esophageal 4 chamber, mid-esophageal long axis, mid-esophageal bi-caval, trans-gastric mid-short, descending thoracic aorta short axis, upper esophageal aortic arch long axis, mid-esophageal ascending aorta short axis, mid-esophageal aortic valve short axis, mid-esophageal right ventricular inflow-outflow, mid-esophageal ascending aorta long axis, upper esophageal aortic arch short axis, and descending aorta long axis; evaluating for intra arrest pathology and adjusting chest compressions as necessary to optimize blood flow, and initiating other treatments to stabilize the patient.

2. The method of claim 1 further comprising three additional views wherein the patient is in shock, wherein the additional views are Mid-esophageal 5 chamber, Mid-esophageal 2 chamber, and Deep trans-gastric 5 chamber.
